(19) United States
(12) Patent Application Publication
(10) Pub. No.: US 2022/0336094 A1
(43) Pub. Date: Oct. 20, 2022

(54) ASSISTIVE SYSTEM USING CRADLE

(71) Applicant: 1THEFULL PLATFORM LIMITED, Seoul (KR)

(72) Inventor: Seung-Yub KOO, Gyeonggi-do (KR)

(21) Appl. No.: 17/639,797

(22) PCT Filed: Jul. 27, 2020
(86) PCT No.: PCT/KR2020/009842

§ 371 Date: Mar. 2, 2022

(30) Foreign Application Priority Data
Sep. 6, 2019 (KR) 10-2019-0110547

(51) Int. Cl. G16H 40/67; G03H 1/04; G03H 1/00; G06T 13/40; H04B 1/3877; H04M 1/04; G06F 1/16

(52) U.S. Cl. CPC G16H 40/67; G03H 1/04; G03H 1/0005; G06T 13/40; H04B 1/3877; H04M 1/04; G06F 1/163

(57) ABSTRACT

An assistive system according to an embodiment of the present disclosure includes a cradle module including a holder unit and a driving unit to rotate the holder unit, and a user terminal placed in the holder unit and controlling the driving unit by outputting a driving control signal. It is possible to provide an assistive service at a low cost without any assistive robot. In addition, it is possible to execute the assistive service by simply placing the user terminal in the cradle module without any manipulation, thereby providing convenience of use, and it is possible to efficiently provide a user with output information outputted through the cradle module and the user terminal, and provide an environment just like having conversation with a real character when providing the assistive service, thereby improving intimacy and reducing loneliness in the user.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR 2020/009842, filed Jul. 27, 2020, which claims priority to the benefit of Korean Patent Application No. 10-2019-0110547 filed in the Korean Intellectual Property Office on Sep. 6, 2019, the entire contents of which are incorporated herein by reference.

### BACKGROUND

### 1. Technical Field

The present disclosure relates to an assistive system using a cradle.

### 2. Background Art

Recently, with the growing use of smart speakers or assistive robots used as artificial intelligence (AI) secretary devices, users are conveniently using them, for example, inquiring about news, weather information and other useful information with voices, asking for music and getting the music, doing online shopping with voices, and controlling Internet of Things (IoT) domestic appliances and lightings remotely by voices.

Meanwhile, more recently, there is a dramatic increase in the number of older people who live alone, but it is not easy for them to purchase high cost smart speakers or assistive robots, and even though high cost smart speakers or assistive robots are purchased, installing and using is complicated, which makes it difficult to efficiently and actively help and support older people who live alone through the smart speakers and assistive robots.

Accordingly, there is a need for affordable assistive robots for efficiently helping and supporting older people who live alone at a low cost.

### SUMMARY

The present disclosure is designed to provide an assistive service through a user terminal and a cradle module in which the user terminal is placed and charged, thereby providing the assistive service at a low cost without any assistive robot.

The present disclosure is further designed to execute the assistive service by simply placing the user terminal in the cradle module without any manipulation, thereby providing convenience of use.

To solve the above-described technical problem, an assistive system using a cradle according to the present disclosure includes: a cradle module including a holder unit and a driving unit to rotate the holder unit; and a user terminal which is placed in the holder unit and controls the driving unit by outputting a driving control signal s1.

In addition, the user terminal includes: a location analysis unit to analyze an input direction of input information i1 including at least one of voice information i1-1 or image information i1-2 of a user, and output user information i2 including at least one of direction information i2-1 or face information i2-2 of the user; and a driving control unit to receive the user information i2 and output the driving control signal s1 based on the user information i2.

In addition, the driving unit includes a first driving unit which is operated by a first driving control signal s1-1 based on the direction information i2-1 of the user in the driving control signal s1 to rotate the holder unit in a direction in which the user is located.

In addition, the driving unit includes a second driving unit which is operated by a second driving control signal s1-2 based on the face information i2-2 of the user in the driving control signal s1 to rotate the holder unit so that the holder unit faces the user's face.

In addition, the driving control unit includes: a rotation control unit to control the driving unit to rotate the holder unit in a direction in which the user is located, by outputting a first driving control signal s1-1 based on the direction information i2-1 of the user in the user information i2; and an angle control unit to control the driving unit so that the holder unit faces the user's face, by outputting a second driving control signal s1-2 based on the face information i2-2 of the user in the user information i2.

In addition, the assistive system using a cradle further includes an assistant server unit to receive a terminal connection signal s2 from the cradle module and apply a service execution signal s3 to the user terminal to execute at least one preset assistive service through the user terminal, when the preset user terminal is placed in the holder unit.

In addition, the assistant server unit includes an avatar generation unit to output and apply an avatar generation signal s4 to the user terminal to output a dynamic graphical image g of a preset character through an image output unit of the user terminal, when the assistant server unit applies the service execution signal s3 to the user terminal.

In addition, the cradle module includes a hologram generation unit to output a hologram image by projecting the dynamic graphical image g outputted through the image output unit of the user terminal.

In addition, the assistive system using a cradle includes a wearable module which is worn on the user's body to receive input of biological information i3 of the user and transmit the biological information i3 to the assistant server unit.

According to the present disclosure, it is possible to provide the assistive service through the affordable user terminal and the cradle module in which the user terminal is placed and charged, thereby providing the assistive service at a low cost without any assistive robot.

In addition, it is possible to execute the assistive service by simply placing the user terminal in the cradle module without any manipulation, thereby providing convenience of use.

Furthermore, it is possible to efficiently provide a user with output information (for example, the assistive service) outputted through the cradle module and the user terminal, and provide an environment just like having conversation with a real character when providing the assistive service, thereby improving intimacy and reducing loneliness in the user.

### BRIEF DESCRIPTION OF THE DRAWINGS

FIG. 1 is a block diagram of an assistive system using a cradle according to an embodiment of the present disclosure.

FIG. 2 is a perspective view showing a user terminal and a cradle module according to an embodiment of the present disclosure.

FIG. 3 is an exploded perspective view of the cradle module of FIG. 2.

FIG. 4 is a diagram showing an internal structure of the cradle module of FIG. 2.

FIG. 5 is a diagram showing the output of a dynamic graphical image of a preset character as a hologram image through the cradle module of FIG. 2.

### DETAILED DESCRIPTION

Hereinafter, some embodiments of the present disclosure will be described in detail through the exemplary drawings. It should be noted that in adding the reference signs to the elements of each drawing, like reference signs denote like elements as far as possible even though they are indicated on different drawings. Additionally, in describing the present disclosure, when a certain detailed description of relevant known elements or functions is determined to obscure the subject matter of the present disclosure, the detailed description is omitted.

Furthermore, in describing the elements of the present disclosure, the terms ‘first’, ‘second’, A, B, (a), (b), and the like may be used. These terms are only used to distinguish one element from another, and the nature of the corresponding element or its sequence or order is not limited by the term. It should be understood that when an element is referred to as being “connected”, “coupled” or “linked” to another element, it may be directly connected or linked to other element, but intervening elements may be “connected”, “coupled” or “linked” between each element.

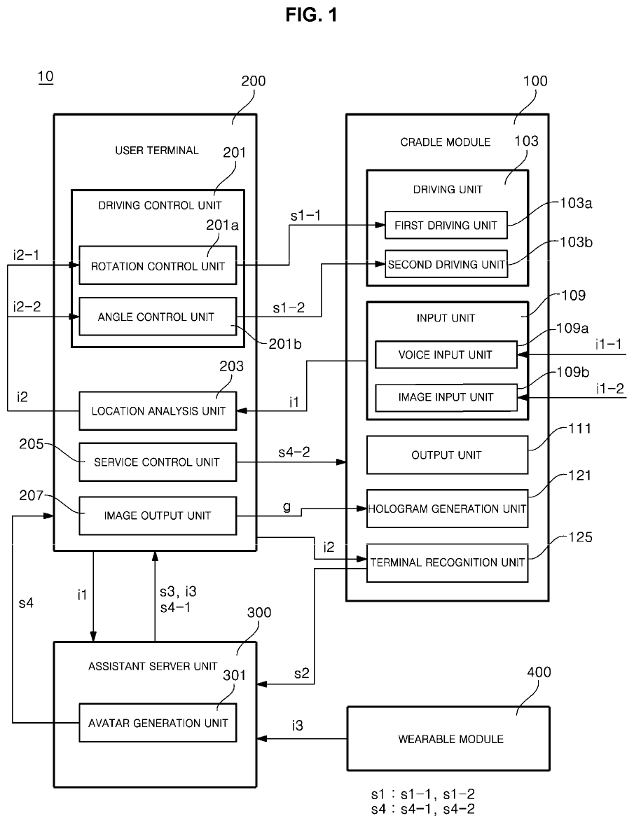

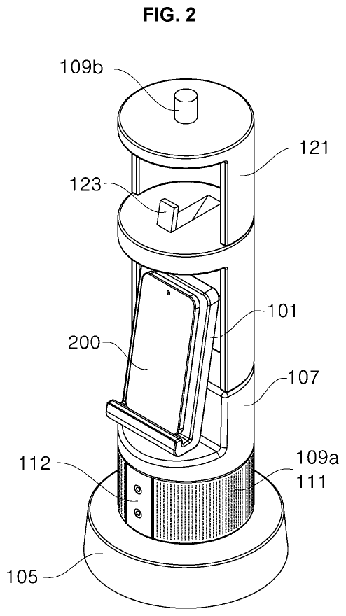

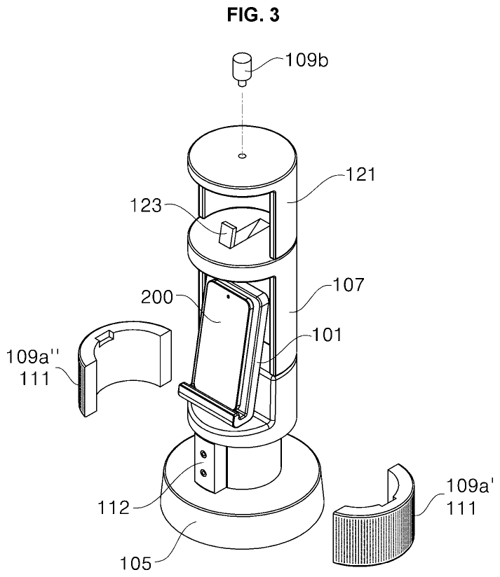

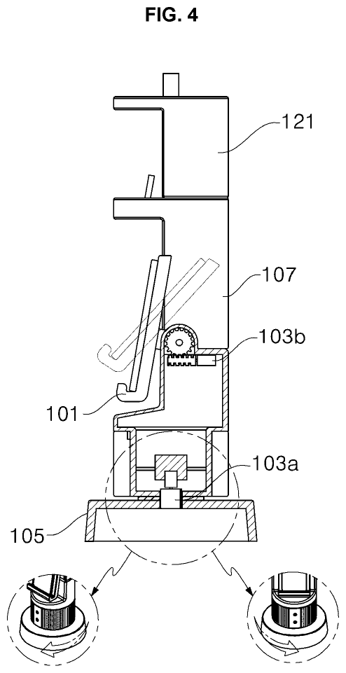

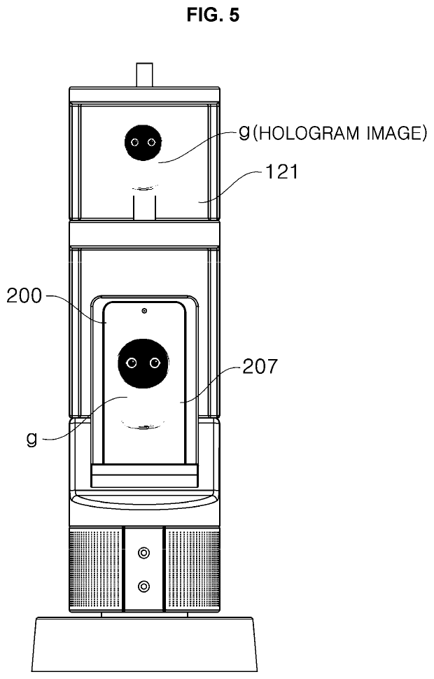

FIG. 1 is a block diagram of an assistive system using a cradle according to an embodiment of the present disclosure. FIG. 2 is a perspective view showing a user terminal and a cradle module according to an embodiment of the present disclosure. FIG. 3 is an exploded perspective view of the cradle module of FIG. 2. FIG. 4 is a diagram showing an internal structure of the cradle module of FIG. 2. FIG. 5 is a diagram showing the output of a dynamic graphical image of a preset character as a hologram image through the cradle module of FIG. 2.

As shown in the drawings, the assistive system 10 using a cradle according to an embodiment of the present disclosure includes: a cradle module 100 including a holder unit 101 and a driving unit 103 to rotate the holder unit 101; and a user terminal 200 which is placed in the holder unit 101 and controls the driving unit 103 by outputting a driving control signal s1.

The cradle module 100 includes the holder unit 101 in which the user terminal 200 is placed, and when the user terminal 200 is placed in the holder unit 101, wired/wireless charging of the user terminal 200 may be enabled.

In this instance, the cradle module 100 may transmit and receive various information and signals to/from the user terminal 200 through a communication unit (not shown) capable of wired/wireless communication, provided in a body unit 107.

The cradle module 100 includes the driving unit 103 which is operated by the driving control signal s1 of the user terminal 200 as described above and rotates the holder unit 101 to control the direction of the user terminal 200.

Describing the structure of the driving unit 103 in further detail, the driving unit 103 includes a first driving unit 103a which is operated by a first driving control signal s1-1 based on the direction information i2-1 of the user in the driving control signal s1 to rotate the holder unit 101 in a direction in which the user is located.

Here, the first driving unit 103a is provided in a base unit 105, and is operated by the first driving control signal s1-1 to rotate the body unit 107 in a direction of rotation of an imaginary axis perpendicular to the bottom, in order to rotate the holder unit 101 and the user terminal 200 in the direction in which the user is located.

Here, the direction information i2-1 of the user is information corresponding to the coordinates at which the user is located, and may be derived through voice information i1-1 or image information i1-2 in input information i1 inputted through an input unit 109 as described below.

Additionally, the driving unit 103 further includes a second driving unit 103b which is operated by a second driving control signal s1-2 based on the face information i2-2 of the user in the driving control signal s1 to rotate the holder unit 101 so that the holder unit 101 faces the user's face.

The second driving unit 103b is provided in the body unit 107, and is operated by the second driving control signal s1-2 to control the angle of the holder unit 101 with respect to the bottom so that the user terminal 200 faces the user's face.

Here, the face information i2-2 of the user is information corresponding to an angle at which the user's face is located, and may be derived through the image information i1-2 inputted through an image input unit 109b.

Meanwhile, the cradle module 100 includes the base unit 105, the body unit 107 rotatably connected to the base unit 105, and the holder unit 101 connected to the body unit 107 at an adjustable angle.

Additionally, the cradle module 100 includes the input unit 109 which receives input of the input information i1 including at least one of the voice information i1-1 or the image information i1-2 of the user, an output unit 111 which outputs an assistive service in the form of a voice or an image, and a switch unit 112.

Here, the input unit 109 includes a voice input unit 109a which receives input of the voice information i1-1 of the user, and the image input unit 109b which receives input of the image information i1-2 of the user.

Here, a plurality of voice input units 109a may be provided in the body unit 107, and for example, the voice input units 109a include a first voice input unit 109a′ provided on one side of the body unit 107 and a second voice input unit 109a″ provided on the other side of the body unit 107.

As the first voice input unit 109a′ and the second voice input unit 109a″ are provided on one side and the other side of the body unit 107, respectively, the level (decibel) of the input voice information i1-1 may be differently inputted depending on the direction in which the user is located, and thereby, the user terminal 200 may detect the input direction of the input information i1 through the level of the input voice information i1-1.

Meanwhile, the first voice input unit 109a′ and the second voice input unit 109a″ may be detachably connected to the body unit 107, and may transmit and receive various voice information i1-1 to/from the user terminal 200 through the communication unit (not shown), and for example, when separated from the body unit 107, the first voice input unit 109a′ and the second voice input unit 109a″ may transmit and receive various voice information i1-1 to/from the user terminal 200 via near-field communication (for example, Wi-Fi, Bluetooth, etc.) through the communication unit.

Subsequently, the image input unit 109b receives input of the image information i1-2 of the user, and the image input unit 109b may be provided as a 360° camera to receive input of the image information i1-2 of the user disposed at various locations with respect to the body unit 107.

Here, in the same way as the voice input unit 109a, the image input unit 109b may be detachably connected to a hologram generation unit 121 provided on the body unit 107, and may transmit and receive various image information i1-2 to/from the user terminal 200, and for example, when separated from the hologram generation unit 121, the image input unit 109b may transmit and receive various image information i1-2 to/from the user terminal 200 via near-field communication (for example, Wi-Fi, Bluetooth, etc.) through the communication unit.

Subsequently, the output unit 111 outputs the assistive service in the form of a voice or an image.

The output unit 111 may be provided as, for example, a speaker integrally formed with the voice input unit 109a.

Subsequently, the switch unit 112 is provided in the body unit 107 and transmits a service ON/OFF signal outputted by the user's manipulation to the user terminal 200, and in this instance, the assistive service as described below may be ON/OFF.

Meanwhile, the cradle module 100 according to an embodiment of the present disclosure further includes the hologram generation unit 121 to output a hologram image by projecting the dynamic graphical image g outputted through an image output unit 207 of the user terminal 200.

The hologram generation unit 121 may be, for example, provided on the body unit 107, and outputs the hologram image by projecting the dynamic graphical image g outputted through the image output unit 207 of the user terminal 200.

The hologram generation unit 121 includes, for example, a projection unit (not shown) to project the dynamic graphical image g outputted through the image output unit 207 to output the hologram image, and a reflection unit 123 to reflect the dynamic graphical image g outputted through the image output unit 207 to project the dynamic graphical image g onto the projection unit.

Here, the projection unit (not shown) may be provided in a polypyramid shape to project the dynamic graphical image g to output the hologram image.

Meanwhile, the cradle module 100 according to an embodiment of the present disclosure further includes a terminal recognition unit 125.

The terminal recognition unit 125 stores terminal information of the initially registered user terminal 200, and when the initially registered user terminal 200 is placed and the terminal information i2 is applied, outputs and transmits a terminal connection signal s2 to an assistant server unit 300 as described below.

Here, the initially registered user terminal 200 may be the user terminal 200 of a model released and supplied by a specific communication company.

Subsequently, the user terminal 200 is placed in the holder unit 101 of the cradle module 100, and when placed in the holder unit 101, the user terminal 200 may transmit and receive various information and signals to/from the cradle module 100 via wired/wireless communication, and the user terminal 200 may be, for example, a smartphone or a tablet PC.

Describing the structure of the user terminal 200 in further detail, the user terminal 200 includes: a location analysis unit 203 to analyze the input direction of the input information i1 including at least one of the voice information i1-1 or the image information i1-2 of the user and output user information i2 including at least one of direction information i2-1 or face information i2-2 of the user; and a driving control unit 201 to receive the user information i2 and output the driving control signal s1 based on the user information i2.

The location analysis unit 203 receives the voice information i1-1 and the image information i1-2 of the user from the input unit 109 of the cradle module 100, outputs the user information i2 including the direction information i2-1 and the face information i2-1 of the user through the voice information i1-1 and the image information i1-2 of the user, and applies it to the driving control unit 201.

In an example, the location analysis unit 203 receives the voice information i1-1 inputted through the plurality of voice input units 109a, compares the level (for example, decibel) of each voice information i1-1 applied from the plurality of voice input units 109a, determines that the user is located in the direction of the voice input unit 109a through which the voice information i1-1 of the largest value is inputted, and outputs the direction information i2-1 of the user.

In another example, the location analysis unit 203 may receive the image information i1-2 from the image input unit 109b, derive a user area in which the image of the user was captured in the image information i1-2, and output the direction information i2-1 of the user.

Meanwhile, the location analysis unit 203 may output the face information i2-2 of the user through the input information i1, and more specifically, the location analysis unit 203 may derive the face information i2-2 of the user through the image information i1-2 inputted through the image input unit 109b in the input information i1.

For example, the location analysis unit 203 may derive the area in which the image of the user was captured in the image information i1-2, and output an area including the user's eyes, nose and mouth in the area in which the image of the user was captured as the face information i2-2 of the user.

Subsequently, the driving control unit 201 receives the user information i2, and controls the driving unit 103 of the cradle module 100 by outputting the driving control signal s1 based on the user information i2.

Describing the structure of the driving control unit 201 in further detail, the driving control unit 201 includes: a rotation control unit 201a to control the driving unit 103 to rotate the holder unit 101 in the direction in which the user is located, by outputting the first driving control signal s1-1 based on the direction information i2-1 of the user in the user information i2; and an angle control unit 201b to control the driving unit 103 so that the holder unit 101 faces the user's face, by outputting the second driving control signal s1-2 based on the face information i2-2 of the user in the user information i2.

The rotation control unit 201a controls the first driving unit 103a to rotate the holder unit 101 in the direction in which the user is located, by outputting the first driving control signal s1-1 based on the direction information i2-1 of the user outputted through the location analysis unit 203.

The angle control unit 201b controls the second driving unit 103b to adjust the angle of the holder unit 101 with respect to the bottom so that the holder unit 101 faces the user's face, by outputting the second driving control signal s1-2 based on the face information i2-2 of the user outputted through the location analysis unit 203.

Meanwhile, the user terminal 200 further includes the image output unit 207, and the image output unit 207 may output a variety of image information and text information.

When the image output unit 207 receives an avatar generation signal s4 from the assistant server unit 300 as described below, the image output unit 207 outputs the dynamic graphical image g of the preset character.

As described above, an embodiment of the present disclosure may derive the direction information i2-1 and the face information i2-2 of the user through the voice information i1-1 and the image information i1-2 of the user, rotate the holder unit 101 in the direction in which the user is located by controlling the first driving unit 103a through the direction information i2-1 of the user, and control the holder unit 101 to face the user's face through the face information i2-2 of the user, thereby improving the transmission efficiency of the output information (for example, the assistive service) outputted through the cradle module 100 and the user terminal 200.

Meanwhile, an embodiment of the present disclosure includes the assistant server unit 300 to receive the terminal connection signal s2 from the cradle module 100 and apply a service execution signal s3 to the user terminal 200 to execute at least one preset assistive service through the user terminal 200, when the preset user terminal 200 is placed in the holder unit 101.

Here, the assistive service is a service for helping and supporting the user, for example, an older adult who lives alone, through an artificial intelligence (AI) chatbot function, and specifically, the assistive service includes services, for example, analysis of the user's pattern provided through the voice information i1-1 and the image information i1-2 of the user inputted through the cradle module 100, a friendship function for inducing the user to have active conversation, video calling, medication reminders, exercise recommendations and detection of the user's motion.

The assistant server unit 300 includes an AI chatbot to transmit and receive a variety of information and signals to/from the user terminal 200 and the cradle module 100 via wired/wireless communication and execute the above-described assistive service.

The assistant server unit 300 receives the voice information i1-1 and the image information i1-2 of the user inputted to the cradle module 100 through the user terminal 200 and analyzes the user's pattern, and when it is determined that the user feels lonely, the assistant server unit 300 actively provides the friendship function and the video calling service, and reminds medication at a preset time and recommends exercises to the user.

In this instance, when multiple users are detected through the image information i2-2 of the user, the assistant server unit 300 may not output the friendship function service, and when the user at risk is detected, may transmit a danger detection signal to a terminal (not shown) of a family member or friend preset as the user's relationship.

When the terminal connection signal s2 is applied, the assistant server unit 300 transmits the service execution signal s3 to the user terminal 200, and applies the service control signal s4 to the user terminal 200 to output the above-described various assistive services through the user terminal 200 or the cradle module 100.

In this instance, the user terminal 200 directly outputs the assistive service (for example, the video calling service, the text service, etc.), or a service control unit 205 transmits the service control signal s4-2 to the cradle module 100 to output the assistive service through the cradle module 100.

Additionally, when the user terminal 200 outputs the service control signal s4-2 to output the specific assistive service, the user terminal 200 controls the driving unit 103 by outputting the driving control signal s1 to efficiently provide the assistive service to the user.

For example, when the voice information i1-1 ‘Dasom, make a video call to my son’ is inputted, the assistant server unit 300 transmits the service control signal s4-1 to the user terminal 200 to allow the user terminal 200 to make a video call using a specific phone number, and in this instance, the user terminal 200 controls the first driving unit 103a to rotate in the direction in which the user is located by outputting the first driving control signal s1-1, and controls the second driving unit 103a to face the user's face by outputting the second driving control signal s1-2.

Also, in case that the assistive service other than the video calling service is outputted, obviously, the user terminal 200 controls the driving unit 103 of the cradle module 100 by outputting the driving control signal s1 to easily provide the assistive service to the user.

Meanwhile, the assistant server unit 300 includes an avatar generation unit 301 to output and apply the avatar generation signal s4 to the user terminal 200 to output the dynamic graphical image g of the preset character through the image output unit 207 of the user terminal 200, when the assistant server unit 300 applies the service execution signal s3 to the user terminal 200.

The avatar generation unit 301 outputs and transmits the avatar generation signal s4 to the user terminal 200, and in this instance, the user terminal 200 outputs the dynamic graphical image g, and the dynamic graphical image g outputted through the user terminal 200 is reflected on the reflection unit 123, projected onto the projection unit (not shown) and outputted as a hologram image.

Here, the dynamic graphical image g of the character outputted as the hologram image may be outputted as an image just like having conversation with the user when the assistive service is outputted.

As described above, an embodiment of the present disclosure provides an environment just like having conversation with a real character when providing the assistive service, thereby improving intimacy and reducing loneliness in the user who lives alone.

Subsequently, the assistive system 10 using a cradle according to an embodiment of the present disclosure further includes a wearable module 400 which is worn on the user's body to receive input of the user's biological information i3 and transmit the biological information i3 to the assistant server unit 300.

The wearable module 400 is worn on the user's body to receive the input of the biological information i3 including the heartbeat, the blood pressure, the body temperature and the blood sugar level and transmit the biological information i3 to the user terminal 200, and in this instance, the assistant server unit 300 may provide the assistive service, for example, medication reminders and exercise recommendations, through the biological information i3.

As described above, according to the present disclosure, since the assistive service is provided through the affordable user terminal 200 and the cradle module 100 in which the user terminal 200 is placed and charged, it is possible to provide the assistive service at a low cost without any assistive robot.

Additionally, since the assistive service is executed by simply placing the user terminal 200 in the cradle module 100 without any manipulation, it is possible to provide convenience of use.

Additionally, it is possible to efficiently provide the user with the output information (for example, the assistive service) outputted through the cradle module 100 and the user terminal 200 and provide an environment just like having conversation with a real character when providing the assistive service, thereby improving intimacy and reducing loneliness in the user.

While the preferred embodiments of the present disclosure have been hereinabove illustrated and described, the present disclosure is not limited to the above-described particular preferred embodiments, and it is obvious to those having ordinary skill in the technical field pertaining to the present disclosure that various modifications and changes may be made thereto without departing from the key subject matter of the appended claims and such modifications fall in the scope of the appended claims.

1. An assistive system, comprising:
a cradle module including a holder unit and a driving unit to rotate the holder unit; and
a user terminal which is placed in the holder unit and controls the driving unit by outputting a driving control signal (s1).

2. The assistive system according to claim 1, wherein the user terminal includes:
a location analysis unit to analyze an input direction of input information (i1) including at least one of voice information (i1-1) or image information (i1-2) of a user, and output user information (i2) including at least one of direction information (i2-1) or face information (i2-2) of the user; and
a driving control unit to receive the user information (i2) and output the driving control signal (s1) based on the user information (i2).

3. The assistive system according to claim 2, wherein the driving unit includes:
a first driving unit which is operated by a first driving control signal (s1-1) based on the direction information (i2-1) of the user in the driving control signal (s1) to rotate the holder unit in a direction in which the user is located.

4. The assistive system according to claim 2, wherein the driving unit includes:
a second driving unit which is operated by a second driving control signal (s1-2) based on the face information (i2-2) of the user in the driving control signal (s1) to rotate the holder unit so that the holder unit faces the user's face.

5. The assistive system according to claim 2, wherein the driving control unit includes:
a rotation control unit to control the driving unit to rotate the holder unit in a direction in which the user is located, by outputting a first driving control signal (s1-1) based on the direction information (i2-1) of the user in the user information (i2); and
an angle control unit to control the driving unit so that the holder unit faces the user's face, by outputting a second driving control signal (s1-2) based on the face information (i2-2) of the user in the user information (i2).

6. The assistive system according to claim 1, wherein comprises:
an assistant server unit to receive a terminal connection signal (s2) from the cradle module and apply a service execution signal (s3) to the user terminal to execute at least one preset assistive service through the user terminal, when the preset user terminal is placed in the holder unit.

7. The assistive system according to claim 6, wherein the assistant server unit includes:
an avatar generation unit to output and apply an avatar generation signal (s4) to the user terminal to output a dynamic graphical image (g) of a preset character through an image output unit of the user terminal, when the assistant server unit applies the service execution signal (s3) to the user terminal.

8. The assistive system according to claim 7, wherein the cradle module includes:
a hologram generation unit to output a hologram image by projecting the dynamic graphical image (g) outputted through the image output unit of the user terminal.

9. The assistive system according to claim 6, wherein comprises:
a wearable module which is worn on the user's body to receive input of biological information (i3) of the user and transmit the biological information (i3) to the assistant server unit.
